(19) United States
(12) Patent Application Publication
(10) Pub. No.: US 2011/0257669 A1
(43) Pub. Date: Oct. 20, 2011

(54) DISPOSABLE LANCET

(75) Inventor: Libo Zhang, Tianjin (CN)

(73) Assignee: TIANJIN HUAHONG MEDICAL CO., LTD., Tianjin (CN)

(21) Appl. No.: 13/142,269

(22) PCT Filed: Mar. 30, 2010
(86) PCT No.: PCT/CN2010/000404

§ 371 Date: Jun. 24, 2011

(30) Foreign Application Priority Data
Apr. 17, 2009 (CN) 200920096338.6

(51) Int. Cl. A61B 17/34

(52) U.S. Cl. 606/182

(57) ABSTRACT

The present invention relates to a disposable lancet. The present invention relates to a technical field of medical appliance. A disposable lancet comprises a needle tube and a needle body disposed in the needle tube. An inner wall of the needle tube is provided with a sliding groove adapted for the sliding of the needle body. A front end of the sliding groove is provided with a locating arris, one end of the needle body is connected with a spring while the other end is detachably provided with a telescopic sleeve. The front end of the needle body is provided with a wedge surface, the telescopic sleeve is provided with a slope surface corresponding to the wedge surface of the needle body, and, the telescopic sleeve is located at a side of the locating arris after the telescopic sleeve has equipped with the needle body. When in use, the needle body is rotated to the other side of the locating arris as the telescopic sleeve is pressed to push the needle body to move to the end of the locating arris, so as to expose the needle tip out of the front end of the needle tube by means of the spring. The present invention has the advantages of compact in structure, easy in use, and simple in manufacture technology. Also, the present invention has less requirements on the equipments, greater reduction on the cost, large-scale manufacture and production and applications.

### FIELD OF THE INVENTION

The present invention relates to a technical field of medical appliance, and in particular, to a disposable lancet.

### DESCRIPTION OF THE RELATED ART

Nowadays, there are various blood taking devices in the markets. Blood taking pen is a relative advanced blood taking device, which may be used in an easy and quick manner. When in use, the needle body of the blood taking pen is disposable while the pen holder may be repeatedly used. However, the repeatedly used pen holder may easily bring cross infection, which exists great safety troubles. Meanwhile, the conventional disposable lancet has the disadvantages of complex in structure, difficult to manufacture, high in production cost, inconvenience in use, and bad in safety.

### SUMMARY OF THE INVENTION

The present invention has been made to overcome or alleviate the problems existing in the prior art. Accordingly, it is an object of the present invention to provide a disposable lancet.

A disposable lancet, comprising a needle tube and a needle body disposed in the needle tube, characterized in that, an inner wall of the needle tube is provided with a sliding groove adapted for the sliding of the needle body, a front end of the sliding groove is provided with a locating arris, one end of the needle body is connected with a spring while the other end is detachably provided with a telescopic sleeve, the front end of the needle body is provided with a wedge surface, the telescopic sleeve is provided with a slope surface corresponding to the wedge surface of the needle body, the telescopic sleeve is located at a side of the locating arris after the telescopic sleeve has equipped with the needle body, when in use, the needle body is rotated to the other side of the locating arris as the telescopic sleeve is pressed to push the needle body to move to the end of the locating arris, so as to expose the needle tip out of the front end of the needle tube by means of the spring.

Preferably, the telescopic sleeve has a needle body sliding opening, the needle body is engaged with the needle body sliding opening when the needle body is rotated to the other side of the locating arris.

Preferably, a needle is provided at the needle body, and a needle tip is provided with a needle cap.

Preferably, a flat head is provided in the front end of the needle cap, the needle cap is disengaged from the needle body by rotating the flat head, the needle tip is exposed by drawing out the needle cap.

Preferably, the spring is provided in such a manner that the needle tip of the needle body is within the needle tube when the spring is in a non-compressed manner.

Preferably, the needle tube has a shape selected from one of a cylindrical shape, a square shape, and a prismatic shape.

Preferably, when the needle tube has a cylindrical shape, said cylindrical outer surface is provided with an anti-rolling surface.

Preferably, the spring is mounted on a cover, and, the cover and the needle tube are mounted together in an interference fit.

As apparent form the above, the present invention has the following advantages and benefits:

The spring of the lancet is almost in a non-compressed state before the lancet is used, so the spring may not be transformed during the long-term store state, so that sufficiency elasticity of the needle tip may be ensured. Since the lancet is without a launch button, it may eliminate mishandling such as unintentional button enablement. Meanwhile, this lancet overcomes secondary uses, and eliminates cross-infections, to greatly improve the safety. The present invention has the advantages of compact in structure, easy in use, and simple in manufacture technology. Also, the present invention has less requirements on the equipments, greater reduction on the cost, large-scale manufacture and production and applications.

### BRIEF DESCRIPTION OF THE DRAWINGS

FIG. 1 is a schematic view of a disposable lancet according to the present invention;

FIG. 2 is a schematic view of a needle tube in FIG. 1;

FIG. 3 is a top view of the needle tube in FIG. 2;

FIG. 4 is a cross-sectional view along line A-A in FIG. 2;

FIG. 5 is a cross-sectional view along line B-B in FIG. 2;

FIG. 6 is a schematic view of a telescopic sleeve in FIG. 1;

FIG. 7 is a top view of a telescopic sleeve in FIG. 6;

FIG. 8 is a schematic view of a needle body;

FIG. 9 is a front view of the needle body in FIG. 8;

FIG. 10 is a top view of the needle body in FIG. 9;

FIG. 11 is a schematic view of a needle body without a needle cap but with a spring;

FIG. 12 is a schematic view of a cover; and

FIG. 13 is a top view of the cover in FIG. 12;

in which:

1—needle tube;

2—telescopic sleeve;

3—needle cap

4—sliding groove

5—locating arris;

6—sliding opening;

7—needle body;

8—wedge surface

9—needle tip

10—spring;

11—cover.

### DETAILED DESCRIPTION OF PREFERRED EMBODIMENTS OF THE INVENTION

The technical solutions and advantages of the present invention will be further described in view of the detailed description of the exemplary embodiments with reference to the accompanying drawings.

### Embodiments

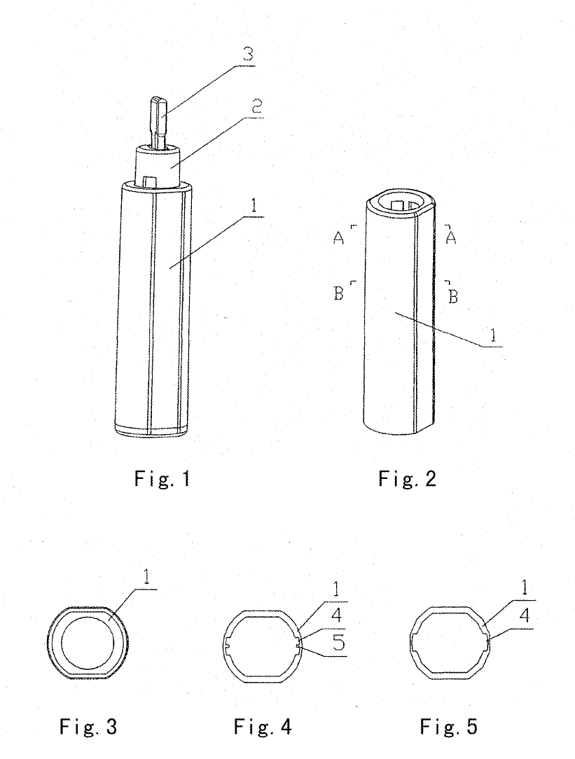

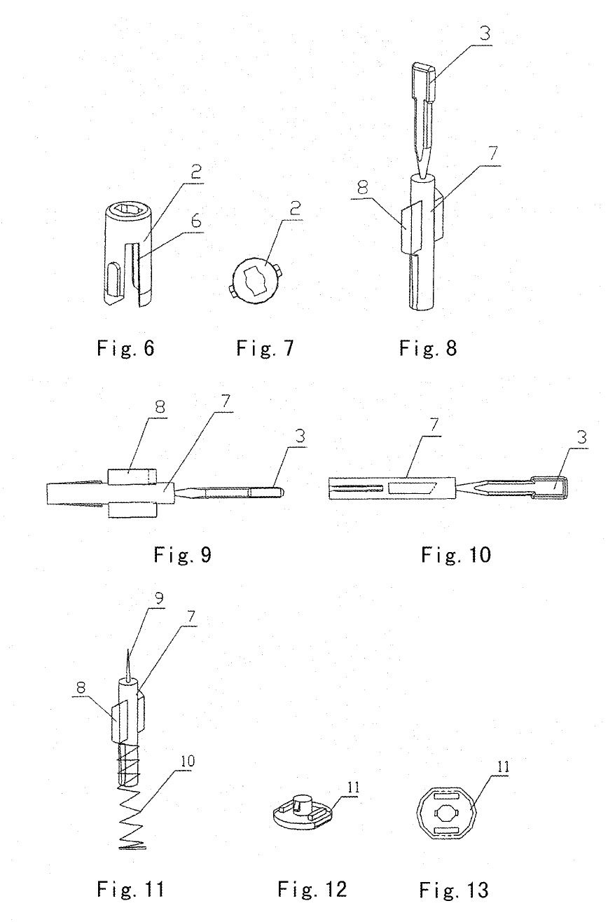

Referring to FIGS. 1-5, 8, a disposable lancet comprises a needle tube 1 and a needle body 7 disposed in the needle tube. An inner wall of the needle tube 1 is provided with a sliding groove 4 adapted for the sliding of the needle body. A front end of the sliding groove 4 is provided with a locating arris 5. As shown in FIGS. 11 and 12, one end of the needle body is connected with a spring 10. The spring 10 is mounted at a cover 11. The cover 11 and the needle tube 1 are mounted together in an interference fit. As shown in FIGS. 1, 6, 8, the other end of the needle body 7 is detachably provided with a telescopic sleeve 2. The front end of the needle body is provided with a wedge surface. The telescopic sleeve 2 is provided with a slope surface corresponding to the wedge surface 8 of the needle body. The telescopic sleeve is located at a side of the locating arris 5 after the telescopic sleeve has equipped with the needle body 7. When in use, the needle body 7 is rotated to the other side of the locating arris as the telescopic sleeve 2 is pressed to push the needle body 7 to move to the end of the locating arris 5, so as to expose the needle tip out of the front end of the needle tube by means of the spring 10. As shown in FIGS. 6-7, the telescopic sleeve 2 has a needle body sliding opening 6, the telescopic sleeve 2 is sleeved on the needle body 7 when the needle body 7 is rotated to the other side of the locating arris.

As shown in FIGS. 9-11, the needle body is provided with a needle, and, the needle tip 9 is provided with a needle cap 3. A flat head is provided in the front end of the needle cap 3, the needle cap is disengaged from the needle body by rotating the flat head, the needle tip 9 is exposed by drawing out the needle cap.

As shown in FIG. 11, the spring is provided in such a manner that the needle tip of the needle body is within the needle tube when the spring is in a non-compressed manner. In the preferred embodiment, the needle tube has a cylindrical shape. Also, the needle tube may have square shape, prismatic shape, or any other suitable shapes.

As shown in FIGS. 1-2, once the needle tube has a cylindrical shape, the cylindrical outer surface is provided with an anti-rolling surface.

As shown in FIGS. 12-13, the spring is mounted on the cover, the cover and the needle tube are mounted together in an interference fit.

Installation and operation of the preferred embodiment

Installation:

The wedge surface of the front end of the needle body is corresponded to the slope surface of the telescopic sleeve. The telescopic sleeve is located at a side of the locating arris after the telescopic sleeve has equipped with the needle body. The needle body pushes the telescopic sleeve to expose the needle tip out of the needle tube by elasticity action of the spring.

Operation:

Twisting off the needle cap to break up the needle cap, and then, pulling out the needle cap to expose the needle tip; aiming the telescopic sleeve at the blood taking site by handling the needle tube, pushing the telescopic sleeve to move the needle body and compress the spring, so that the needle body is rotated to the other side of the locating arris when the needle body is moved to the end of the locating arris, so that the needle body is ejected by the spring and the needle tip is exposed from the front end of the needle tube. The telescopic sleeve 2 has a needle body sliding opening 6. When the needle body 7 is rotated to the other end of the locating arris, the slope surface of the telescopic sleeve 2 is staggered with the wedge surface 8 of the slope surface, and, the wedge surface 8 of the slope surface partially enters the sliding opening 6 of the telescopic sleeve 2, so as to engage the telescopic sleeve 2 with the needle body 7. The needle tip 9 of the needle body 7 is exposed from the telescopic sleeve 2 to achieve the blood taking operation. Since the needle tip of the needle body is within the needle tube when the spring is in a non-compressed manner, the needle tip is retracted into the needle tube after the completion of the blood taking operation. The needle tube has a cylindrical shape, and the cylindrical outer surface is provided with two flat planes so as to effectively prevent the rolling of the blood taking needle and facilitate the disposal and use.

What is claimed is,:

1. A disposable lancet, comprising a needle tube and a needle body disposed in the needle tube, characterized in that, an inner wall of the needle tube is provided with a sliding groove adapted for the sliding of the needle body, a front end of the sliding groove is provided with a locating arris, one end of the needle body is connected with a spring while the other end is detachably provided with a telescopic sleeve, the front end of the needle body is provided with a wedge surface, the telescopic sleeve is provided with a slope surface corresponding to the wedge surface of the needle body, the telescopic sleeve is located at a side of the locating arris after the telescopic sleeve has equipped with the needle body, when in use, the needle body is rotated to the other side of the locating arris as the telescopic sleeve is pressed to push the needle body to move to the end of the locating arris, so as to expose the needle tip out of the front end of the needle tube by means of the spring.

2. The disposable lancet according to claim 1, characterized in that, the telescopic sleeve has a needle body sliding opening, the needle body is engaged with the needle body sliding opening when the needle body is rotated to the other side of the locating arris.

3. The disposable lancet according to claim 1, characterized in that, a needle is provided at the needle body, and a needle tip is provided with a needle cap.

4. The disposable lancet according to claim 3, characterized in that, a flat head is provided in the front end of the needle cap, the needle cap is disengaged from the needle body by rotating the flat head, the needle tip is exposed by drawing out the needle cap.

5. The disposable lancet according to claim 1, characterized in that, the spring is provided in such a manner that the needle tip of the needle body is within the needle tube when the spring is in a non-compressed manner.

6. The disposable lancet according to claim 1, characterized in that, the needle tube has a shape selected from one of a cylindrical shape, a square shape, and a prismatic shape.

7. The disposable lancet according to claim 6, characterized in that, when the needle tube has a cylindrical shape, said cylindrical outer surface is provided with an anti-rolling surface.

8. The disposable lancet according to claim 1, characterized in that, the spring is mounted on a cover, and, the cover and the needle tube are mounted together in an interference fit.
